(19) United States
(12) Patent Application Publication
(10) Pub. No.: US 2023/0012059 A1
(43) Pub. Date: Jan. 12, 2023

(54) METHOD AND DEVICE FOR OBTAINING THE TEMPORAL OLFACTORY SIGNATURE OF A SAMPLE AND USES OF THE METHOD

(71) Applicant: ARYBALLE, GRENOBLE (FR)

(72) Inventors: Romain DUBREUIL, Grenoble (FR); Cyril HERRIER, Grenoble (FR); Thierry LIVACHE, Jarrie (FR); Johanna DECORPS, Four (FR); Yann GUEGANNO, Grenoble (FR)

(21) Appl. No.: 17/757,533

(22) PCT Filed: Dec. 15, 2020
(86) PCT No.: PCT/FR2020/052443

§ 371 Date: Jun. 16, 2022

(30) Foreign Application Priority Data
Dec. 16, 2019 (FR) FR1914518

(51) Int. Cl. G01N 33/00; C11B 9/00; G16C 60/00; G16C 20/70

(52) U.S. Cl. CPC G01N 33/0031; G01N 33/0073; C11B 9/00; G16C 60/00; G16C 20/70

(57) ABSTRACT

The present invention relates to a method for characterising, by means of an electronic nose, the release kinetics of odorous compounds from a sample, comprising the following series of steps: (a) supplying a sample; (b) at a time t1, exposing the sensor array of the electronic nose to some of the gaseous medium comprising the odorous compounds released from the sample, and processing the response emitted by the sensor array of the electronic nose, after said exposure, in the form of a signal; and (c) repeating step (b) at least once, at a time t2 different from the time t1, whereby an olfactory kinetic signature characterising the sample is obtained. The present invention also relates to the use of this method for anti-counterfeiting and/or quality control purposes and for generating a data bank or database of temporal olfactory signatures. The present invention finally relates to certain devices used when implementing such methods.

### TECHNICAL FIELD

The present invention relates to the general technical field of electronic noses.

The present invention more particularly proposes a method for characterizing samples which over time release, produce and/or emit a plurality of odorant compounds, using an electronic nose, and on that basis providing a temporal olfactory signature.

The present invention also relates to the use of this method in anticounterfeiting and/or in quality control within fields where objects or products are made of materials which, initially or over time, emit odorant compounds, such as, in particular, in the field of perfumery, natural or synthetic odorant products, cosmetics, spirits or else plastics, and for generating a bank or database of temporal olfactory signatures.

The present invention lastly relates to certain devices used in the implementation of such methods.

### PRIOR ART

A perfume, or other aromatic compounds such as spirits, is or are composed of a collective of highly varied molecules in liquid phase, which gradually pass from this phase into the gaseous phase.

With regard to perfumes, a phrase used is “the olfactory pyramid”, which corresponds to the three groups of molecules which evaporate over the course of time after unstoppering, vaporization or application, these being the “head note”, the “heart note” and the “base note”.

The head notes consist of the lightest and most volatile primary constituents which also disappear the quickest, at between a few minutes and 2 hours. The duration of this “head note” will vary from one perfume to another, and even from one expert nose to another for the same perfume. The molecules of medium volatility correspond to the “heart notes”, and have a lifetime of 2 to 10 hours. Next come the “base notes”, which sometimes signify the authenticity of a perfume. They continue to be sensed 10 hours after the application of the perfume.

While perfume makers keep their manufacturing formulation secret, there are many perfumes that are counterfeited. The differences between an authentic perfume and a counterfeit lie primarily at the level of the heart notes and the base notes. The reason is that the constituent chemical products of these notes are more complex and more expensive, and hence more difficult for the counterfeiter to copy.

The challenge for those wishing to detect a counterfeit is therefore to be able to detect a perfume which is fake/copied from an original perfume.

Many schools of perfumery train persons endowed with olfactory expertise, also referred to as “noses”, who are able to tell the difference between two odors with very similar chemical compositions. The approach conventionally employed is called a blind triangle measurement. An odor is therefore always sensed in comparison with another odor and never on an absolute basis. As regards the temporal analysis of a perfume, the experts are capable of detecting a counterfeit of a perfume by controlled venting of this perfume. Specifically, the head notes and the heart notes are evaporated in order to enable analysis of the base notes which signify the quality of a perfume. More generally, the comparison of odorant formulas, beyond counterfeiting, remains complicated.

International application WO 2015/181257 A2 [1] describes a technique for evaluating a perfume disposed in a closed, thermally regulated chamber by an expert nose who evaluates an odor intensity measurement, after prior evaporation of the head notes. A spatio-temporal profile of a perfume is defined in an objectivization procedure. A product detection threshold is defined within a perfume by successive dilutions. The olfactometers are instruments specially designed for performing this step and providing a detection limit.

The approaches above each involve a human expert nose who describes and quantifies the odor. The obvious limits of this type of approach are tied to the fact that the expert nose can only carry out a limited number of analyses within a likewise limited time. Furthermore, two expert noses will not necessarily produce the same responses.

International application WO 2017/167407 A1 [2] relates to a device for examining the sillage of the perfume by venting a few microliters thereof (typically 20 μL) on a glass slide in a tube several meters long and by carrying out olfactory controls at various sampling/measurement points. This type of instrument allows the kinetic analysis of a perfume by performing accelerated air dilution of the perfume. The system enables controls to be performed at a number of points in the odor sillage so as to generate a spatio-temporal profile. In this application, the olfactory controls may be performed not only by an expert nose but also by analytical techniques such as microextraction on a solid phase, thermal desorption, or high-field asymmetric ion mobility.

Other complex analytical tools such as selected ion flow tube mass spectrometry or gas chromatography coupled with mass spectrometry are used by expert users. They allow the editing of complex spectra on mixtures which are themselves complex. Although they are employed in quality control by perfume makers, they are not very widely used in the rapid detection of counterfeiting, owing to low availability of time and of qualified personnel. The measurements involved are expensive and require a lot of analysis time. This approach, as described in patent CN105572202 B [3], aims to carry out identification and possible quantification of the chemical compounds present in the perfume. In practice, the identification of the products yields sometimes several hundred products, so making the measurement difficult to utilize. Furthermore, different compositions may generate the same odor. As another drawback, this technique does allow an analysis to be made at a given time, but certainly not an in-depth analysis over time.

Another known alternative is that the electronic noses allow the detection and objective identification of a mixture of volatile compounds and especially of volatile organic compounds (VOC). These instruments are attracting genuine industrial interest for the measurement of odors, especially in association with the objectivization of an odor.

They may be coupled with an automatic sampling of the odor by virtue of an injection system. These systems have already been deployed for comparing signatures obtained between original bottles of perfumes and counterfeit bottles of perfumes. The method presupposes the prior learning of a large number of replicas in order to deploy analytical techniques of support vector machine type [4, 5]. It should be noted that the technique for distinguishing and detecting counterfeit perfumes by using an electronic nose, described in Cano et al., 2011 [4], does not involve a study of the kinetics of release of the odorant compounds. Moreover, obtaining a database allows the signatures measured to be compared for the purpose of classifying the product (compliant or noncompliant). Accordingly, the results are often presented in the form of a principal component analysis, allowing the results to be analyzed in 2 dimensions, usually by evaluating the difference between groups of points or clusters. It is stated, moreover, that the ethanol present in the perfumes interferes with the analysis [5]. Lastly, this type of analysis reveals in particular the most volatile compounds, including the ethanol and the head notes; the less volatile, heavier compounds, especially those of the tail note, remain largely masked by these signals.

This approach is visually satisfactory as it enables an evaluation of the distances between two groups. On the other hand, it employs support vector machine-type signal processing algorithms, leave-one-out algorithms, etc., which do not always allow the origin of the difference to be interpreted.

In Li et al., 2017 [6], the experiments are performed by analysis of a single withdrawal from the sample, of the Chinese liquors type, which makes it possible, indeed, to compare these liquors, but not to compare their temporal development.

In Eklov et al., 1998 [7], release kinetics of odorant compounds are disclosed that are the result of a fermentation, i.e., of a biological and/or enzymatic development, of sausages and not of odorant compounds which are already present in the sample at the time it is supplied. No description is given of any comparison of temporal olfactory signatures. On the contrary: the samples are compared with one another at a given time by plotting the PCAs for each time under consideration (FIGS. 5 to 8). Lastly, the device employed in [7] does not have any means promoting or accelerating the release of the odorant compounds from the sausage samples. In particular, gaseous samples are taken from the beakers containing the sausages, and air is added to these beakers in order to replace the gas withdrawn: consequently there is no forced flow at all.

The “static” approaches proposed in the prior art do not enable temporal analysis or dynamic analysis of the temporal development of the odors. There is, though, a genuine need for such analysis not only in the context of counterfeit identification but also in many other fields, both industrial and otherwise, such as, for example, the quality control in the field of spirits, the quality control of batches of perfumes or else of natural or synthetic odorant products.

### SUMMARY OF THE INVENTION

The present invention is aimed at resolving the technical problems of the prior art and at alleviating their weaknesses. To accomplish this, the present invention proposes a rapid and objective solution for rapidly analyzing the temporal development of a sample such as a complex mixture of odorant compounds.

In contrast to the general prescription in instances where a sample must be stable over time, the inventors are proposing an analytical method which allows the development over time of the odorant components released, produced or emitted by a sample to be monitored.

The present invention more particularly provides a method for characterizing, by means of an electronic nose, the kinetics of release of odorant compounds from a sample, comprising the following successive steps:

a) providing a sample;

b) at a time t₁, exposing the network of sensors of said electronic nose to a portion of the gaseous medium comprising the odorant compounds released from said sample, and processing the response emitted by the network of sensors of said electronic nose, following said exposure, in the form of a signal; and

c) repeating step b) at least once, at a time t₂ different from the time t₁, thereby obtaining a temporal olfactory signature which characterizes said sample.

The term “kinetics of release of the odorant compounds” refers to the release, production and/or emission, over time, of various odorant compounds in gas form from a sample. The expressions “kinetics of release of the odorant compounds”, “kinetics of production of the odorant compounds”, “kinetics of emission of the odorant compounds”, and “kinetics of diffusion of the odorant compounds” are equivalent in the present text and may be used interchangeably.

The term “odorant compound” refers to any compound or any molecule which is detectable by an animal olfactory system and especially the human olfactory system.

The odorant compounds for which the release, production and/or emission are characterized in the context of the present invention may be inorganic compounds or organic compounds. The latter include volatile organic compounds (VOC).

As a reminder, the concept of VOC is defined by Directive 1999/13/EC of the European Council of Mar. 11, 1999, whereby:
a volatile organic compound is “any organic compound having at 293.15 K (or 20° C.) a vapor pressure of 0.01 kPa (or 9.87×10⁻⁵ atm) or more, or having a corresponding volatility under the particular conditions of use” (cf. paragraph 17 of Article 2 of the Directive);
an organic compound is “any compound containing at least the element carbon and one or more of the following elements: hydrogen, halogens, oxygen, sulfur, phosphorus, silicon or nitrogen, with the exception of carbon oxides and inorganic carbonates and bicarbonates” cf. paragraph 16 of Article 2 of the Directive).

VOCs therefore include, especially, certain saturated or unsaturated acyclic hydrocarbons (ethane, propane, n-butane, n-hexane, ethylene, propylene, 1,3-butadiene, acetylene, etc.), certain saturated or unsaturated nonaromatic cyclic hydrocarbons (cyclopropane, cyclopentane, cyclohexane, etc.), certain aromatic hydrocarbons (benzene, toluene, xylenes, ethylbenzene, etc.), certain halogenated hydrocarbons (dichloromethane, trichloromethane, chloroethane, trichloroethylene, tetrachloroethylene, etc.), certain alcohols (methanol, ethanol, 1-propanol, 2-propanol, ethylene glycol, propylene glycol, etc.), certain aldehydes (formaldehyde, acetaldehyde, propanal, 2-propenal (or acrolein), 3-ethoxy-4-hydroxybenzaldehyde, etc.), certain ketones (acetone, methyl ethyl ketone, 2-butanone, methyl vinyl ketone, etc.), certain esters (methyl acetate, ethyl acetate, isopropyl acetate, isoamyl butyrate, etc.), certain ethers (diethyl ether, ethylene glycol n-butyl ether, 1,4-dioxane, etc.), certain acids (acetic acid, propanoic acid, 2-methylundecanoic acid, etc.), certain amines (ethylamine, dimethylamine, trimethylamine, diethylamine, amylamine, etc.), certain amides (dimethylformamide, for example), certain thiols (methyl mercaptan or methanethiol, ethyl mercaptan or ethanethiol, etc.), certain nitriles (acetonitrile, acrylonitrile, etc.), and other compounds comprising a plurality of different chemical functions.

International application WO 2015/181257 A2 [1] cites numerous odorant compounds and especially numerous VOCs that are present in perfumes.

The term “sample” refers in the context of the present invention to any element or composition capable of releasing, producing and/or emitting one or more odorant compounds in vapor phase.

A sample accordingly is a mixture of stable or prestabilized chemical compounds including at least one odorant compound. The qualitative chemical composition of this sample is stable over time (no molecular change), but the proportions of said compound or compounds may develop during the time of the method according to the invention.

The present invention allows the release, production and/or emission of the odorant compounds from a single sample to be monitored over time, using an electronic nose. In other words, the odorant compounds analyzed by virtue of this electronic nose, in the invention, are present in the sample at the time it is supplied and are released, produced and/or emitted at different times depending on their volatility. Accordingly, the evolution by depletion of the odorant compounds in the samples that are the most volatile is monitored, after which the odorant compounds with weaker volatility and possibly intensity are found. The odorant compounds for which the release kinetics are studied/characterized in the context of the present invention, consequently, are not the result of biological, catalytic and/or enzymatic development of the sample during the method of the invention.

The sample to be studied in the context of the present invention may be a solid sample such as, for example, a plastic, cardboard, a cosmetic product, a synthetic odorant product or a synthetic deodorant product such as an automobile odorant-product diffusion strip, etc. As a variant, the sample may be a liquid sample such as, for example, a perfume, a flavor, an essential oil, a soda, a simple spirit drink, also known as a distilled spirit, such as whiskey, or else a composite spirit drink such as a liqueur.

Prior to the implementation of the characterization method according to the invention, the sample may have undergone a treatment. This treatment may be chemical or thermal and may be aimed at stabilizing or, conversely, breaking down the sample. In that case the sample supplied in step a) of the method according to the invention corresponds to the treated sample. An illustrative example would include the addition of an acid to a plastic for the purpose of monitoring, via the characterization method according to the invention, the odorant compounds given off during the breakdown of the plastic as caused by the acid added.

Step a) of the method according to the invention is typically carried out at a time denoted time t₀.

In step a) of the method according to the invention, the sample for which it is desired that the kinetics of release of the odorant compounds are characterized, and especially the liquid sample, may be applied to a solid support. Any solid support may be used in the context of the present invention.

Said support may be a nonporous support such as a glass or metal support. When the sample is liquid, this sample, on a nonporous support of this kind, forms one or more droplets which, relative to the site of application, present a contact angle having a value dependent on the wettability of the support relative to said sample.

As a variant, this support may be porous and may therefore absorb the liquid sample. In that case the porous solid support allows the surface/volume ratio to be maximized, so that the odorant compounds present in the liquid state in this sample receive maximum exposure to the surrounding gas and are able to pass into the gaseous phase in a greater quantity. The use, accordingly, of a porous solid support bearing the applied liquid sample also enables controlled, and especially accelerated, evaporation of the odorant compounds contained in said sample.

Examples of porous supports that may be used in the context of the present invention include synthetic or non-synthetic animal or human skin; paper, especially cellulosic paper; cotton paper; agarose; gelatin; cellulose; methylcellulose; carboxymethylcellulose; nitrocellulose; cellulose acetate ester; an alginate; a polyolefin; a porous membrane such as a Nafion membrane; a Sephadex resin; a polyvinylidene fluoride or PVDF membrane; a polyester resin; a glass fiber fabric; porous glass; a polyacrylamide gel; a sepharose gel; a silica gel; or a mixture thereof. A porous support of this kind may therefore take the form of a plaque, a membrane, a ribbon, a strip, a powder, a cloth, a resin, or else a gel. Certain gels and resins that may be used are known under the name of odor-diffusing polymers.

The solid support used in the context of the invention is advantageously a porous solid support. As a particular example, when the sample is liquid, the porous solid support used is synthetic or non-synthetic animal or human skin; porous glass; or a strip of cellulosic paper in the form of a perfumery smelling strip.

Advantageously, when a solid support is used, it does not interact with the sample and especially with the odorant components produced, released and/or emitted by the sample. Alternatively, it may absorb constituents present in the sample, such as, for example, a silica gel, which may absorb the water present in a sample while permitting the evaporation of the nonhydrophilic odorant components. The solid support may also influence the kinetics of release of the odorant compounds from the sample, such as, for example, a synthetic or non-synthetic animal or human skin.

More particularly, when the sample is liquid, a small amount of this sample, typically less than 1000 μl, especially less than or equal to 500 μL, less than or equal to 200 μL or else less than or equal to 100 μL, is applied to the porous or nonporous support. The use of a small amount of liquid sample allows promoted (or controlled) and especially accelerated evaporation of the odorant components contained in said sample.

The sample, whether solid or liquid, applied to a solid support may be disposed in a container such as a bottle, a test tube, a flask, a cannister, etc. In certain embodiments, this container may be hermetically sealed. As a variant, this container may have one or more openings enabling gas exchanges between its internal volume and the external environment.

Step b) of the method comprises characterizing the odorant compounds already released from the sample at a time t₁ via an electronic nose.

As a reminder, an “electronic nose” is an instrument for detecting target compounds in gaseous phase such as odorant compounds. The electronic nose owes its name to the analogy which exists between its operation and that of the human olfactory system. The electronic nose is made up principally of three systems, these being:

(1) a fluidic system for transporting a gaseous sample from outside the electronic nose into the inside of this nose, said system playing the part of the respiratory system;

(2) a detection system which comprises a network of sensors having cross-reactivity for volatile compounds present in a sample of the gaseous medium, the sensors playing the part of the olfactory receptors of the human nose; and

(3) a computer system performing the processing of the responses emitted by the sensors in the form of signals, this system playing the part of the human brain.

Step b) involves, firstly, contacting the sensors of the electronic nose with a portion of the gaseous medium comprising the odorant compounds released from the sample.

During this contacting and in the case where the sample, liquid or solid, is optionally applied to a solid support and placed in a container, the electronic nose utilized in this contacting may be placed in the same container.

As a variant, this contacting may comprise carrying out a withdrawal from the gaseous medium comprising the odorant compounds released from the sample, and exposing the network of sensors of the electronic nose to this withdrawal.

In this portion of gaseous medium or in this gaseous withdrawal, the odorant compounds already released from the sample are present, and are therefore in the gaseous state in this gaseous medium or in this withdrawal. In fact this gaseous medium is proximate to the sample, i.e., the gaseous medium surrounding the sample, and the withdrawal must be made from a gaseous medium of this kind. The gaseous medium surrounding the sample is conventionally termed the “head space”. When the sample has been placed in a container, this gaseous medium is the gaseous medium in the internal volume of the container. In the case of withdrawal, this withdrawal is typically made from the gaseous medium in the internal volume of the container.

The time interval between the time t₀ and the time t₁ at which, respectively, step a) and step b) are performed may be variable. The reason is that, in order to prevent any loss of information about the most volatile odorant compounds released, produced and/or emitted by the sample, the time t₀ and the time t₁ will be selected to be very close to one another, with the time interval in this case being a few seconds. As a variant, if the information relating to the most volatile odorant compounds released, produced and/or emitted by the sample is not deemed to be necessary, the time interval between the time t₀ and the time t₁ will be much longer and may be as much as several minutes, or even at least an hour or even several hours. This variant may be utilized especially in the case of a perfume sample for which the aim is to evaporate the head note to the air without analysis by the electronic nose of the odorant compounds included in the head note.

When withdrawal from the gaseous medium has taken place, said medium is conveyed via the fluidic system (1) of the electronic nose to the detection system (2) of the nose, in order to expose the network of sensors of the electronic nose to the withdrawal and therefore to the odorant compounds in the gaseous state that it contains.

The sensitive part of the sensors of the electronic nose which interacts with the odorant compounds present in the gaseous medium or in the withdrawal may consist of semiconducting metal oxides, semiconducting polymers, or else may be functionalized with one or more biomolecules, i.e., molecules present naturally in living beings, such as oligonucleotides, nucleic acids, carbohydrates, peptides, proteins, lipids, etc., or biomimetic molecules, i.e., molecules which structurally and/or functionally imitate biomolecules.

Step b) of the method according to the present invention then comprises converting the physicochemical interactions which take place between the sensitive part of the sensors and odorant components in the gaseous state into usable signals.

The sensors present in the detection system of the electronic nose may typically each comprise their own measuring system—or transducer—or may share with other sensors a measuring system which is common to them. In both cases, the measuring system may be any measuring system capable of generating a usable signal during the physicochemical interaction between a compound in the gaseous state and the sensitive part of a sensor, and may especially be a resistive, piezoelectric, mechanical, acoustic or optical system. In other words, the sensors may be resistive, piezoelectric, mechanical, acoustic and/or optical sensors.

The sensors advantageously are optical surface plasmon resonance sensors, interferometric sensors, or else micromachined ultrasound transducer sensors, and more particularly capacitive micromachined ultrasound transducer (or CMUT) or piezoelectric micromachined ultrasound transducer (or PMUT) sensors.

In the case of optical surface plasmon resonance sensors, this type of transduction generally combines a light source, of LED type, for example, so as to give rise to plasmonic excitation, and a CCD camera for recording the signal resulting from the plasmonic resonance. For this purpose, very particular preference is given to monitoring the signals emitted by sensors in imagery mode, which involves monitoring the signal variations of all of the pixels making up the image from the CCD camera used.

Measurement at any time t generates an n-dimensional response, from which a normalized signature can be extracted using techniques such as PCA (principal component analysis), MDS (multidimensional scaling, i.e., multidimensional positioning), and neural networks for obtaining reduced dimensionality. As a particular example, when the electronic nose used is the NeOse Pro from Aryballe Technologies, each measurement at the time t generates a response with 68 dimensions, because of a network of 68 sensors in parallel, and a normalized signature can be obtained on the basis of this response [8, 9].

Step c) of the method according to the invention comprises repeating the substeps of step b) at least at one time t₂, which is different from said time t₁ at which step b) is performed. In other words, step c) allows the odorant compounds in gaseous form that have come from the sample and are present in the environment surrounding said sample to be characterized at a time t₂ via an electronic nose.

Step c) advantageously comprises repeating step b), i.e., the substeps of steps b), at a plurality of separate times. It should be noted that between two consecutive times, t₀ and tₙ₊₁, the withdrawal system is kept in place and the sample undergoes no displacement and no modification other than the release, production and/or emission of odorant compounds.

By “plurality of separate times” is meant more than 2, especially more than 5, in particular more than 10, and more particularly more than 20 separate times. These different times may be selected with regular intervals or with irregular intervals. The measurements are made advantageously at regular intervals via an automated withdrawal system, which promotes the standardization of the method. The skilled person will know how, without inventive effort, to determine the most appropriate time interval depending on the sample to be tested. As a particular example, in the case of a liquid perfume sample, this interval may be between 5 min and 1 h and especially between 10 min and 30 min.

The time interval between the time t₀ and the time t₁ of the last withdrawal and the last analysis in step c) is variable and depends on the sample under study.

The measurements at each of the withdrawal and analysis times, i.e., at least at times t₁ and t₂, produces an n-dimensional response and where appropriate a normalized signature for each of the withdrawal and analysis times. The temporal olfactory signature obtained at the outcome of the method according to the invention therefore corresponds to the entirety of the n-dimensional responses and, optionally, normalized signatures obtained at the outcome of the method according to the invention.

The expressions “temporal olfactory signature”, “olfactory diffusion signature”, “olfactory signature over time”, “dynamic olfactory signature” or “kinetics of olfactory signature” are equivalent and may be used equivalently in the context of the present invention.

Moreover, where the sample is placed in a container, the method according to the invention further comprises a step of introducing air into the container, where this introduction may be concomitant with or consecutive to each withdrawal from the gaseous medium containing the odorant compounds released by the sample, i.e., from the internal volume of the container. It should be noted, however, that this introduction of air is not mandatory for ensuring the implementation of the method according to the invention.

This introduction of air may be obtained simply by compensating for the aspiration accompanying withdrawal, either passively by a vent present in the container level, or actively by a forced ventilation.

Furthermore, during the method according to the invention, the release of the odorant compounds from the sample may be promoted or accelerated. As set out earlier, promoted or accelerated release may be obtained by using a small amount of liquid sample and/or by applying this liquid sample to a porous solid support. Promoted or accelerated release may also be obtained by heating the sample, whether solid or liquid, i.e., by promoting the passing of the liquid compounds into vapor phase and/or by agitating the sample, either solid or liquid. This agitation also ensures a uniform composition of the gaseous medium containing the odorant compounds released from the sample. Similarly, when the sample is placed in a container, the latter may have an unclosed opening which promotes the passage of the volatile products into the air surrounding the container, or else may have a forced flow system for accelerating the renewal of the gaseous medium containing the odorant compounds released from the sample. Accordingly, these volatile products disappear more rapidly from the internal volume of the container from which the various withdrawals are made.

This promoted or accelerated release is advantageous when the aim is to evaporate the most volatile odorant compounds as rapidly as possible and to recover the least volatile odorant compounds more rapidly as well. As a particular example, these more volatile products are especially ethanol, when the sample is a liquid perfume or a spirit. Similarly, this promoted or accelerated release is advantageous in respect of counterfeit perfumes which contain essentially head notes, which are often a product of less expensive compounds, in contrast to the authentic perfumes, which differ from the former in their content of less volatile odorant compounds which are often more expensive.

The present invention also relates to a method for comparing two samples E1 and E2 and especially for comparing the kinetics of release of odorant compounds from two samples E1 and E2. This comparison method comprises
characterizing the kinetics of release of the odorant compounds from the sample E1 by a characterization method as defined above and obtaining a temporal olfactory signature which characterizes said sample E1;
characterizing the kinetics of release of the odorant compounds from the sample E2 by a characterization method as defined above and obtaining a temporal olfactory signature which characterizes said sample E2;
comparing the temporal olfactory signature which characterizes said sample E1 and the temporal olfactory signature which characterizes said sample E2.

It is obvious that the operating conditions of the steps of characterizing the kinetics of release of the odorant compounds from the samples E1 and E2 must be identical. Examples of such operating conditions include the same quantity (volume or mass) of samples E1 and E2; identical solid supports to which the samples E1 and E2 may be applied; identical conditions of temperature and/or humidity in the characterizing steps; withdrawals and analyses at identical times; electronic noses having identical features, and especially the same electronic nose; same procedure for converting the physicochemical interactions which take place between the sensitive portion of the sensors and the odorant compounds in the gaseous state into usable signals; and/or same procedure for converting the usable signals into a normalized signature.

It is also possible, however, to contemplate the use of this comparison method for studying conditions liable to influence the kinetics of release, production or emission of the odorant compounds from a sample. In that scenario, sample E1 and sample E2 are identical, while at least one of the operating conditions listed above is different. For example, the solid support to which sample E1 is applied is different from the support used for sample E2, or else the amounts of sample E1 and sample E2 are different.

In one embodiment, the step of characterizing the kinetics of release of the odorant compounds from the sample E1 and the step of characterizing the kinetics of release of the odorant compounds from the sample E2 are performed simultaneously. In this embodiment, the two characterizing steps, i.e., from sample E1 and from sample E2, may be carried out on electronic noses which are different but exhibit identical characteristics. As a variant, these two characterizing steps may be performed using the same electronic nose. In this variant and in practice, at a given time, sample E1 is contacted with the network of sensors of the electronic nose used, after which the latter is rinsed before being contacted with sample E2. The time difference between the two contacting procedures is minimal in light of the interval between the times t₀ and tₙ₊₁ as defined above whereby the samples E1 and E2 have substantially the same “age” when these two contacting procedures take place.

In another embodiment, the step of characterizing the kinetics of release of the odorant compounds from the sample E1 and the step of characterizing the kinetics of release of the odorant compounds from the sample E2 are separated in time. This embodiment is used especially when the step of characterizing the kinetics of release of the odorant compounds from one of the two samples has been performed beforehand and recorded in a database.

As set out above, the electronic nose may be placed in the container accommodating the solid or liquid sample, optionally placed on a solid support. In such an instance, the method of comparison may employ two electronic noses, each placed in a container. One of the two containers comprises sample E1, and the other sample E2. The comparison of the two samples is based on the value of the difference between the two electronic noses. It is not mandatory for the sensors of each of the electronic noses to be rinsed; the flow of air which enables aging ensures the ventilation of the sample and of the sensors of each of the electronic noses. As a variant, each of the electronic noses may be removed from its container, whereby its network of sensors is rinsed by the ambient air.

The step of comparing the temporal olfactory signature characterizing said sample E1 and the temporal olfactory signature characterizing said sample E2 uses processes that are highly conventional in the analysis of data in electronic noses.

As set out above, each measurement at time t for each of samples E1 and E2 generates an n-dimensional response from which it is possible to extract a normalized signature. It is possible accordingly to construct a distance between 2 normalized signatures. For a set of measurements, therefore, a distance matrix is obtained which describes all of the distances between all of the measurements made. This distance matrix forms the basis for the algorithm of MDS, which enables two-dimensional visualization of a distance matrix much larger in size.

As a variant, the comparison of the signatures of olfactory kinetics may comprise plotting only the absolute value of the distances as a function of the time. Accordingly, from the measurements obtained, it is possible to plot the curves of distances between the two samples E1 and E2.

In the context of the comparison method according to the present invention, a decision threshold may be used for the purpose of comparing the temporal olfactory signature of sample E1 with that of sample E2. For this purpose, for a given time, a calculation is made of the distance between the signature of sample E1 and that of sample E2. If this distance is greater than a threshold value, the two signatures are different and hence the two samples E1 and E2 are different. Conversely, the signatures and hence the two samples E1 and E2 will be deemed to be identical if the distance is less than the threshold value.

In accordance with normal distribution, almost all of the values are situated in an interval centered around the mean, with limits situated 3 standard deviations either side of said mean. This threshold value (vs) may therefore correspond to the following formula: vₛ=3σ, where σ represents the standard deviation or reliability of the electronic nose used in the characterizing steps. This standard deviation or reliability of the electronic nose may become more valuable through the performance of at least two measurements on the same type of sample at a given time t.

In practical terms, the procedure is as illustrated in FIG. 1. Accordingly, at a given time t, two standard samples are measured, and from the measurements a mean of index y and a standard deviation σ are calculated. At the same time t, the sample under test is measured, and its distance/its difference relative to the mean of index of the two standards is measured. A decision is made: “compliant” if δ<3σ or “not compliant” if δ>3σ.

This simple technique employs the simple calculation of a distance between the current sample and the mean of two standards measured simultaneously or beforehand, in order to determine if the two samples are considered to be different in terms of temporal olfactory signatures. In the case of bioinspired electronic noses, this index y may be a raw or normalized signature, i.e., a geometric response combination of a plurality of sensors.

The comparison method as defined above may be used for studying conditions liable to influence the kinetics of release of odorant compounds from a sample. In that case the sample E1 will be a sample not subjected to the test condition, whereas the sample E2 has been or is subjected to this condition. This condition may be an aging time, a physical or chemical treatment likely to degrade this sample, or else a physical or chemical treatment likely to stabilize this sample. This use may be applied, as illustrative and nonlimiting examples, in the field of plastics processing and of packaging and especially to samples made of plastic or of cardboard.

The comparison method as defined above may be used in anticounterfeiting. In this use, the sample E1 may be a liquid sample such as a perfume, a flavor, an essential oil, a simple spirit beverage or a composite spirit beverage, and the sample E2 may be a liquid sample in respect of which a desire is to verify whether it is identical to the sample E1 or whether, conversely, it is a counterfeit of that sample. The present invention accordingly proposes a rapid and reliable anticounterfeiting method which may be performed on site such as, for example, in the customs zone of an airport.

The comparison method as defined above may be used in quality control in the fields of perfumery, essential oils, flavors, synthetic odorant products, synthetic deodorant products, or spirits. In this use, the sample E1 may be a liquid sample such as, for example, a reference perfume, and the sample E2 is the same liquid sample but obtained from a different production batch.

It should be noted that everything which has been set out with regard to the comparison of the two samples E1 and E2 applies to the comparison of more than two separate samples and especially to the comparison of 3, 4, 5, 6, 7, 8, 9 and 10 different samples, or even to the comparison of more than 11 different samples.

The present invention also relates to a method for generating a bank (or database) of temporal olfactory signatures, which comprises
characterizing the kinetics of release of the odorant compounds from a plurality of samples by a method as defined above and obtaining a plurality of temporal olfactory signatures which characterize said samples;
from the plurality of temporal olfactory signatures which characterize said samples, obtained in the preceding step, generating a bank (or database) of temporal olfactory signatures.

A “plurality of samples” refers to at least 2, at least 5, at least 10, and optionally at least 50 samples as defined above.

Bank (or database) generation may comprise recording the plurality of temporal olfactory signatures which characterize said samples and also the conditions used for characterizing the kinetics of release of the odorant compounds, on a suitable computer medium.

The present invention relates lastly to a device which may be used in the implementation of the characterization method, of the comparison method and/or of the generation method as have been defined above.

The device according to the invention comprises

(i) at least one container as defined above, in which a sample as defined above is disposed or may be placed;

(ii) an electronic nose having a fluidic system capable of transporting the gaseous medium comprising the odorant compounds released from said sample to the detection system of the electronic nose, which comprises a network of sensors with cross-reactivity for the odorant components present in said gaseous medium, and a computer system carrying out the processing of the responses emitted by the sensors in the form of signals;

(iii) a means for processing the signals generated by the computer system of said electronic nose so as to obtain a temporal olfactory signature; and

(iv) a means for promoting or accelerating the release of the odorant compounds from the sample.

The means (iv) of the device according to the invention may take the form of an agitator plate and/or hot plate, a heating system, an unclosed opening traversing said container and/or a forced flow system for accelerating the renewal of the gaseous medium containing the odorant compounds released from the sample.

In the device according to the invention, the electronic nose (ii) may be placed in the container (i). As a variant, the device according to the invention may comprise a means for withdrawing from said container the gaseous medium comprising the odorant compounds released from said sample, said means being fluidically connected to the fluidic system of said electronic nose (ii).

At the fluidic connection between the withdrawal means and the fluidic system of the electronic nose, a valve may be installed which is programmed so as to pump, at determined times, the gaseous medium comprising the odorant compounds released from the sample, i.e., the gaseous medium present in the internal volume of the container.

The device according to the invention advantageously comprises a plurality of containers in which identical or different samples as defined above are disposed or may be placed. More particularly, said device comprises at least two, at least three, at least four, at least five, at least six or else at least seven containers in which identical or different samples as defined above are disposed or may be placed.

The device according to the invention may further have a container to which a reference stable over time has been added or may be added. This reference is composed of a pure volatile product such as, for example, butanol diluted in a nonvolatile phase. Moreover, the concentration of this reference is sufficient to generate a constant head space throughout the time needed for the analysis. The reference therefore acts as an internal control in the device according to the invention and during the various methods according to the invention.

The number of withdrawal means in the device according to the invention matches the number of containers. These withdrawal means are advantageously connected fluidically to a multiway valve, which is itself connected fluidically with the fluidic system of the electronic nose. This multiway valve is programmed so as to pump the gaseous medium present in the internal volume of the various containers at determined times.

When the device according to the invention has a single container or multiplicity of different containers, said container(s) may be placed on a support, such as, for example, a plate, or else an agitating plate and/or hot plate.

The device according to the invention may also have a means for introducing air into the internal volume of the container, concomitantly with or consecutively to each withdrawal from this volume. This means may take the form of a vent present in the container, for passive introduction, or the form of a forced ventilation, for active introduction.

The device according to the invention, lastly, may have a means for rinsing the network of sensors of the electronic nose after each contact of said nose with a withdrawal. Such rinsing, carried out advantageously with a neutral gas such as ambient air, dry air, humid air of controlled humidity, helium, dinitrogen, argon or carbon dioxide, enables removal of any gaseous withdrawal from the network of sensors.

Other features and advantages of the present invention will become apparent to the skilled person from a reading of the examples below, which are given for the purpose of illustration and are nonlimiting, with reference to the appended figures.

### BRIEF DESCRIPTION OF THE FIGURES

FIG. 1 already described the development of an index y as a function of time, with y defining a sum or a product of differences or a cosine (similarity index for a scalar product of vectors). Two known standards are measured at each time, from which a mean and a standard deviation are calculated. By measuring the sample for comparison at the same time t, it is possible to calculate a difference δ from the mean of the indices of the two known standards. At times t₁ to t₃, therefore, δ<3σ, and the samples are therefore compliant. At the time t₄, a non-compliance is measured, since δ>3σ.

FIG. 2 is a schematic representation of a device according to the present invention.

FIG. 3 shows the comparison of three perfumes (A, B and C) by successive withdrawals (labeled 0 to 17) at high frequency on a Neose Pro electronic nose from Aryballe Technologies. MDS analysis enables a two-dimensional representation of the evolution of complex signatures having 68 dimensions [8].

FIG. 4A shows the 3D representation of a replica “a” of an original perfume “L”.

FIG. 4B shows the 3D representation of a replica “a” of a copy of the perfume “L”, copy “LL”.

FIG. 4C shows the 3D representation of a replica “b” of the perfume “L”.

FIG. 4D shows the 3D representation of a replica “b” of the copy “LL”.

FIG. 5 represents the development, over time, of the distance between samples which are two duplicates of the same authentic perfume (TrueR1 and TrueR2) and a copy of this perfume (FalseR1). This visualization technique enables a decision to be made on the basis of a criterion of type 3σ.

FIG. 6A shows the temporal diffusion, for a reference perfume, with two types of aging: normal (A and B) and accelerated (C).

FIG. 6B shows the temporal diffusion, for a test perfume, with two types of aging: normal (A and B) and accelerated (C).

FIG. 7A shows the 3D representation of a replica “a” of the original perfume “L” with normal aging.

FIG. 7B shows the 3D representation of a replica “b” of the perfume “L” with normal aging.

FIG. 7C shows the 3D representation of a replica “c” of the perfume “L” with accelerated aging.

FIG. 8A shows the 3D representation of a replica “a” of a copy of the perfume “L”, copy “LL”, with normal aging.

FIG. 8B shows the 3D representation of a replica “b” of the copy “LL” with normal aging.

FIG. 8C shows the 3D representation of a replica “c” of the copy “LL” with accelerated aging.

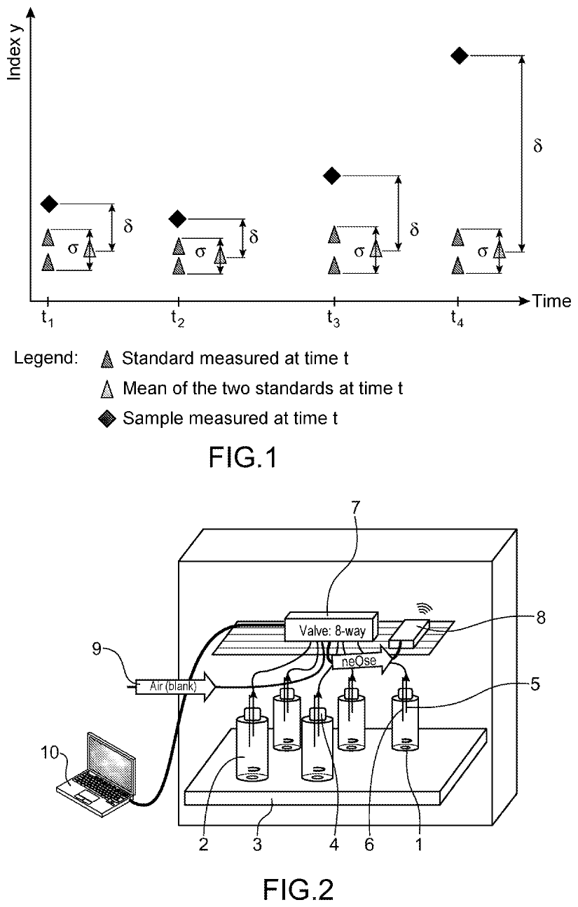

### DESCRIPTION OF CERTAIN EMBODIMENTS

### I. Example of a Device Usable in the Context of the Invention

FIG. 2 shows a device which can be used in the context of the present invention.

The samples 1 for analysis, which are liquid or solid, are placed in containers such as bottles 2 (five are represented in FIG. 2). The liquid samples are applied beforehand to solid supports as defined above, which are placed in the bottles 2. These bottles are disposed on a magnetic stirring plate 3 intended to agitate them and, in so doing, to agitate the solid samples or the solid supports bearing the liquid samples, so as to promote evaporation of the odorant compounds contained or produced and to homogenize the composition of the head space and advantageously of the gaseous medium contained in each bottle 2.

The bottles 2 are closed with stoppers 4. Each stopper 4 has a septum which contains at least two through-holes. At the first of these holes, the means for withdrawing the headspace 5 from the internal volume of the bottle 2 is introduced. This withdrawal means 5 takes the form of a hollow needle or a tube. At the second opening in the septum, the means for introducing air 5 into the internal volume of the bottle 2, concomitantly with or consecutively to each withdrawal from said volume, is introduced. This means 6 takes the form of a vent such as a hollow needle or a tube.

Each withdrawal means 5 is connected fluidically to a multiway valve 7 such as an eight-way valve. This multiway valve 7 is also connected fluidically to the NeOse Pro electronic nose 8 from Aryballe Technologie, and more particularly to the fluidic system of such an electronic nose 8. The multiway valve 7 is connected fluidically, furthermore, to a source 9 of a neutral gas, such as ambient air, which is used for rinsing the sensor network of the electronic nose 8 between two contacting procedures with withdrawals.

The operation of the multiway valve 7 is controlled by a computer 10 so as to withdraw, at determined times, the gaseous medium present in the internal volume of each of the bottles 1, to inject each withdrawal into the electronic nose 8, and lastly, after each injection of a withdrawal, to rinse the network of sensors of the electronic nose 8 with the neutral gas.

### II. Differentiation of Three Perfumes in Terms of Temporal Olfactory Signatures

The objective is to study the development of the olfactory notes of three separate perfumes, denoted perfumes A, B and C. This study was carried out using a multiple sampler and a stirring plate.

50 μl of sample are withdrawn and applied to the end of a smelling strip. Each strip is left in the open air for 30 seconds, and then 7.5 cm of the strip, comprising the end to which the sample has been applied, are inserted into a 50 ml brown bottle. The other end is placed on the thread, and hence on screwing, the cap holds the strip in suspension in the bottle.

The stopper is formed by a septum allowing the introduction of two stainless steel needles, one enabling the withdrawal of the headspace and the other acting as a vent for renewal of the air in the bottle on each withdrawal.

Each sample is analyzed in duplicate, i.e., two strips bearing a single sample are placed in two separate bottles. Analysis by multidimensional scaling (MDS) enables a two-dimensional representation of the development of complex signatures having 68 dimensions [8].

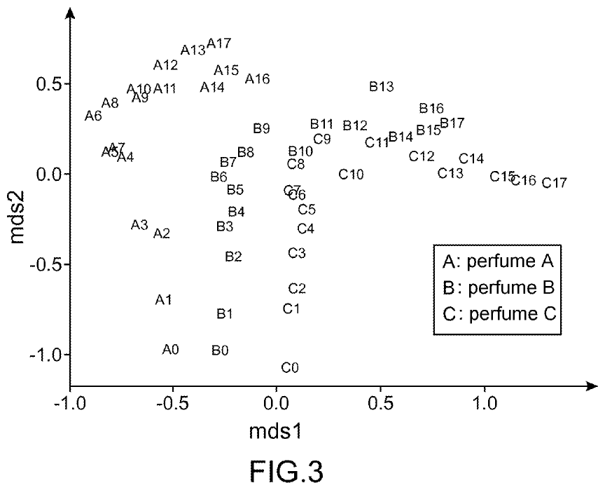

The results are shown in FIG. 3, where each point corresponds to the two-dimensional value obtained for the complex signature of a given perfume on a given withdrawal or injection. Thus the point A12 corresponds to the two-dimensional value obtained for the 12th withdrawal of perfume A.

For the first withdrawal, the perfumes are very close, owing in particular to the ethanol remnant. After 14 to 17 withdrawals, the three perfumes are correctly separated.

### III. Qualitative Analysis of a Perfume and of a Copy in Terms of Temporal Olfactory Signatures

The objective is a qualitative analysis of two perfumes through observation of the development of their notes. The focus is on an original perfume (perfume L) and a copy of said perfume (perfume LL).

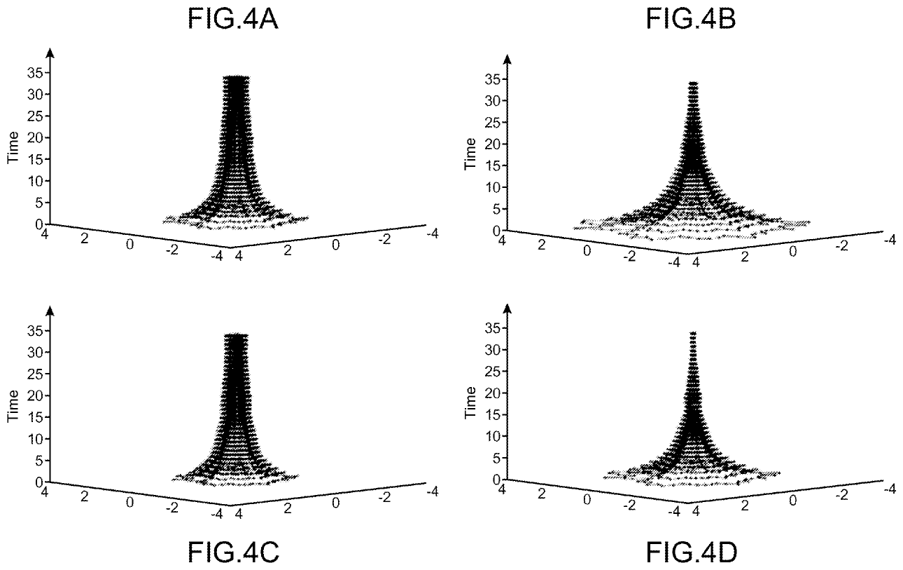

The protocol the same as that used in section II above was implemented with two replicas, for the perfume and its copy. The results, however, are presented in a different form, specifically as an olfactory tunnel for a perfume (3D). The form presented in FIG. 4 enables a very rapid visualization of the difference between the perfume and its copy.

Very readily observable, on the olfactory tunnels of the perfume (FIGS. 4A and 4C), are the base notes of the perfume, corresponding to a greater signal amplitude at long times, especially after time 25. On the olfactory tunnels of the perfume copy (FIGS. 4B and 4D), the intensity is much weaker for these same long times.

### IV. Differentiation Based on Development of the Distance of Measurements on Different Samples, Made by the Electronic Nose

Each measurement at time t generates an n-dimensional response, with 68 dimensions in the case of the NeOse Pro [8], from which a normalized signature can be extracted. It is therefore possible to construct a distance between two normalized signatures, this being very much a conventional process of data analysis in electronic noses.

For a set of measurements, accordingly, a matrix of distances is obtained which describes all of the distances between all of the measurements made. This matrix of distances forms the basis for the MDS algorithm, enabling two-dimensional visualization of a distance matrix much larger in size.

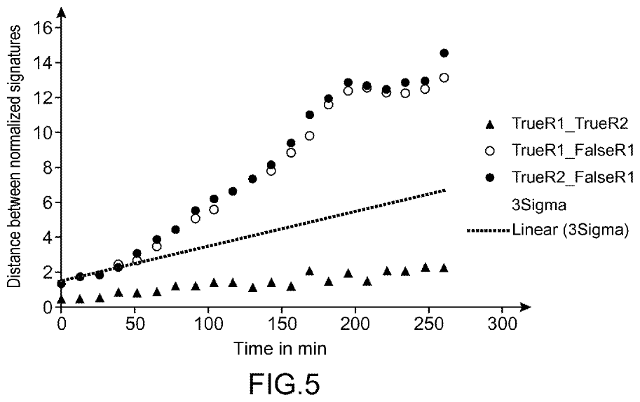

The other technique involves plotting only the absolute value of the distances as a function of time. In FIG. 5, the distance between the duplicates of the same authentic perfume (TrueR1 TrueR2) is represented over time and corresponds to the measurement of two known standards of perfume. This distance represents the standard deviation, or the reliability of the instrument for the same type of samples at the time t. By convention, three times this distance is selected as the decision threshold. Beyond the distance 3σ, the sample is considered to be different from the two standards measured.

Accordingly, from the measurements obtained, it is possible to plot the distance curves between the copy (FalseR1) and each of the two duplicates of the reference perfume (TrueR1 and TrueR2). These two curves correspond to the plots TrueR1 FalseR1 and TrueR2 FalseR1. It is found that below the time t=50 min, these two curves are contained in the interval 3σ described above. Conversely, after this time, the two curves become more distant and are beyond the defined threshold. According to this example, therefore, it may be stated that the counterfeit is detectable after 50 min for these given samples.

### V. Acceleration of the Temporal Diffusion of a Perfume

The accelerated aging of the perfumes is performed using a hotplate on which the bottles containing the perfumes are placed, or by imposing a headspace renewal stream, whereas the normal, i.e., nonaccelerated, aging of the perfumes is performed in the absence of a hotplate or without a headspace renewal stream. The volumes of liquid analyzed are identical in all the bottles.

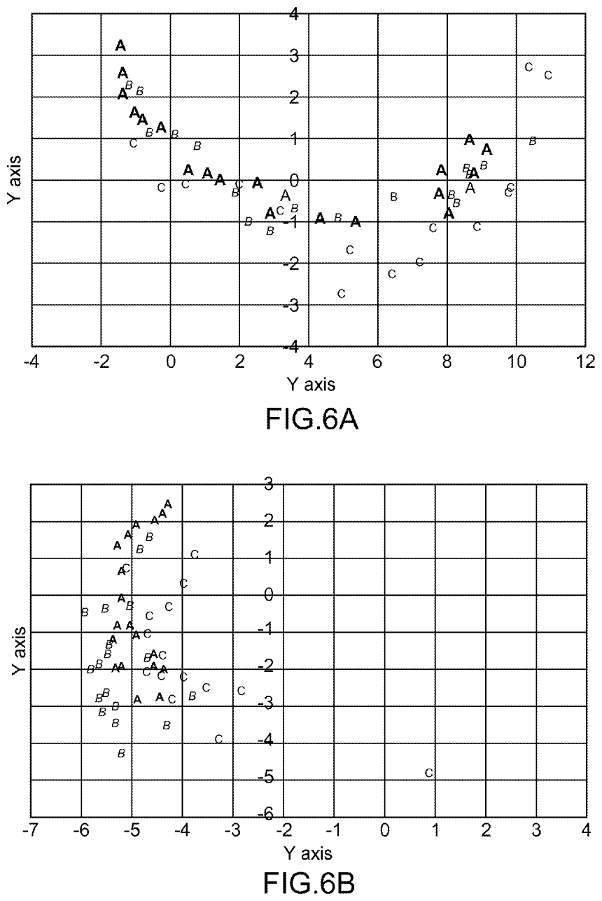

The results are given in the form of an MDS representation in FIG. 6. In this figure, it is seen that the temporal diffusion of the samples of perfume or of counterfeit perfume in the pierced bottles with septum (points C in FIGS. 6A and 6B, respectively) are accelerated, since the signatures develop more quickly over time, compared with those obtained for the perfume duplicates or counterfeit perfume duplicates in the unpierced bottles with septum (points A and B in FIGS. 6A and 6b, respectively).

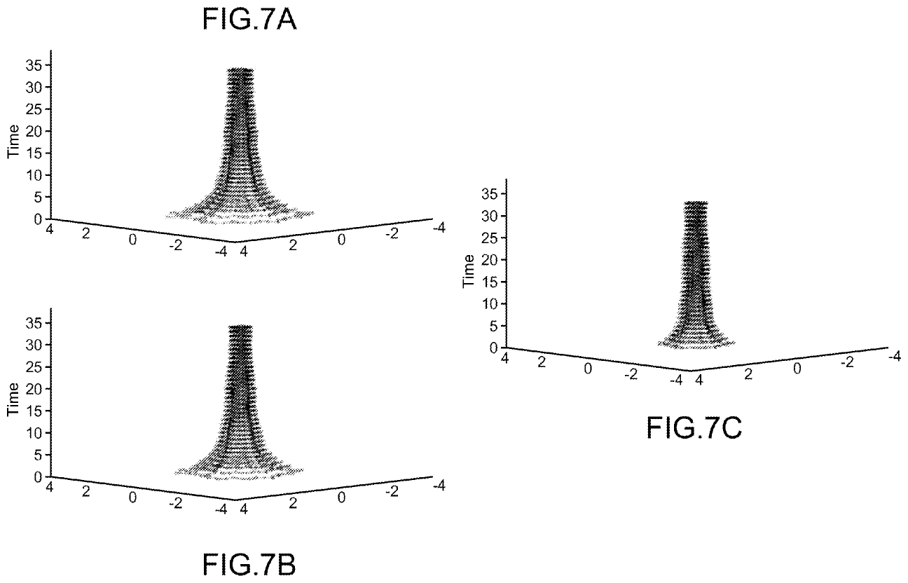

FIG. 7 represents the comparison of the olfactory diffusion in the case of three replicas (a, b and c) of the perfume “L”, of which replicas a and b are subjected to standard aging (FIGS. 7A and 7B) and the replica c to accelerated aging (FIG. 7C). The replica c of the perfume L is quicker to stabilize with regard to the base notes. Between times 10 and 15, the replica is already well stabilized in terms of the base notes, whereas replicas a and b are still developing significantly. Over long times, conversely, the intensity is still significant, which may be interpreted as the presence of the base notes in this perfume.

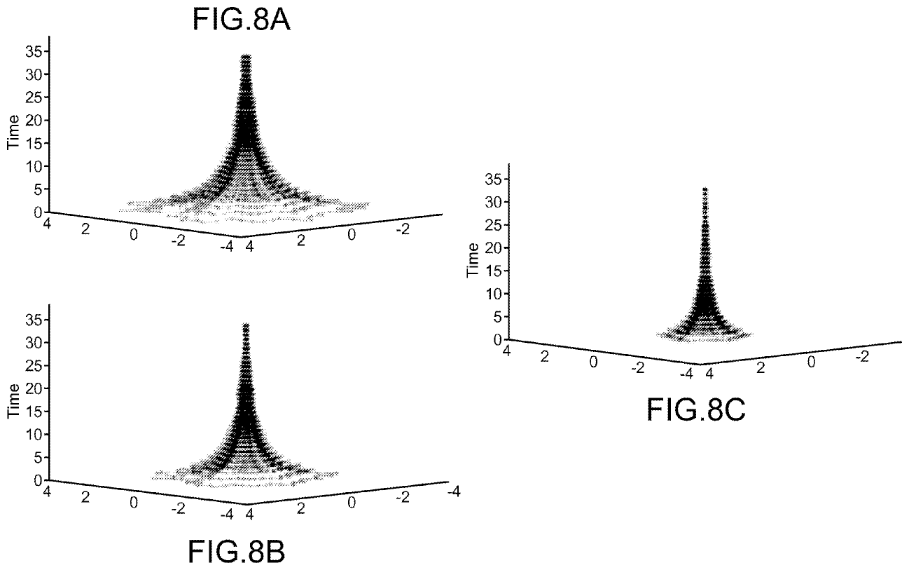

FIG. 8 represents the comparison of the olfactory diffusion in the case of 3 replicas (a, b and c) of a perfume copy “LL”, of which replicas a and b are subjected to standard aging (FIGS. 8A and 8B) and replica c to accelerated aging (FIG. 8C). In this example, the replica c of the perfume copy “LL” is less intense from the start, and the intensity reduces very rapidly. Over long times, the intensity is virtually zero.

### VI. Differentiation of Two Whiskeys in Terms of Olfactory Signatures

Equipment:

For analysis and comparison of two whiskeys, an 8-way valve, 50 ml brown glass bottles equipped with a stopper with septum, magnetic stirrers, a stirring plate, and paper smelling strips were used. The experimental scheme is the same as before; the NeOse Pro instrument from Aryballe Technologies is set for “dynamic measurement” with a cycle of 180 seconds.

### Sample Preparation:

The samples analyzed are two whiskeys C and B. In parallel, a 40% vol/vol ethanol solution is likewise used. For the analysis, 100 μl of sample are applied to a paper smelling strip, the strip is left in the open air for 30 seconds, and then the end bearing the sample is placed in a bottle, with the other end being held on the thread, after which the stopper is screwed over the strip. The whiskeys are prepared in duplicate.

The vent used is a 1.20×40 mm Sterican® needle. The 6.5 cm PTFE tube is brought into each bottle. Additionally, the bottles are placed under slow stirring for 15 min before analysis is commenced.

### BIBLIOGRAPHIC REFERENCES

[1] International patent application WO 2015/181257 A2
[2] International patent application WO 2017/167407 A1
[3] Patent CN105572202 B
[4] Cano et al., Sensors & Actuators: B. Chemical 2011, 156, 319-324
[5] Gebicki et al., Measurement Science and Technology 2015, 26, 125103
[6] Li et al., J. Sensors 2017, 17, 272
[7] Eklöv et al., J. Sci. Food Agri 1998, 76, 525-532
[8] Brenet et al., Analytical Chemistry 2018, 90, 9879-9887
[9] Brenet et al., ISOCS/IEEE International Symposium on Olfaction and Electronic Nose 2017.

1. A method for characterizing, by means of an electronic nose, kinetics of release of odorant compounds from a sample, comprising the following successive steps of:
a) providing a sample;
b) at a time t₁, exposing a network of sensors of said electronic nose to a portion of the gaseous medium comprising the odorant compounds released from said sample, and processing the response emitted by the network of sensors of said electronic nose, following said exposure, in the form of a signal; and
c) repeating step b) at least once, at a time t₂ different from the time t₁, thereby obtaining a temporal olfactory signature which characterizes said sample,
wherein said odorant compounds characterized for their release kinetics are not the result of biological, catalytic and/or enzymatic development of the sample during said method, and
wherein the release of said odorant compounds from said sample is promoted or accelerated by using a sample quantity of less than 1000 μl, by applying the sample to a porous solid support, by heating the sample, by agitating the sample and/or by using a container in which the sample is placed that has an unclosed opening or a forced flow system.

2. The method of claim 1, wherein said sample is a solid sample.

3. The method of claim 1, wherein said sample is a liquid sample applied to a solid support.

4. The method of claim 3, wherein said solid support is synthetic or non-synthetic, animal or human skin; porous glass; or a cellulosic paper strip in the form of a perfumery smelling strip.

5. The method of claim 1, wherein said sample or said solid support is placed in a container.

6. The method of claim 1, wherein step c) comprises repeating said step b) at a plurality of separate times.

7. The method of claim 5, further comprising a step of introducing air into the container, wherein said introduction may be concomitant with or consecutive to each withdrawal from the gaseous medium containing the odorant compounds released by the sample.

8. A method for comparing the kinetics of release of odorant compounds from two samples E1 and E2, comprising the steps of
characterizing the kinetics of release of the odorant compounds from the sample E1 by the method of claim 1, and obtaining a temporal olfactory signature which characterizes said sample E1;
characterizing the kinetics of release of the odorant compounds from the sample E2 by the method of claim 1, and obtaining a temporal olfactory signature which characterizes said sample E2; and
comparing the temporal olfactory signature which characterizes said sample E1 and the temporal olfactory signature which characterizes said sample E2.

9. The comparison method of claim 8, wherein said step of characterizing the kinetics of release of the odorant compounds from the sample E1 and said step of characterizing the kinetics of release of the odorant compounds from the sample E2 are performed simultaneously.

10. The comparison method of claim 8, wherein said step of characterizing the kinetics of release of the odorant compounds from the sample E1 and said step of characterizing the kinetics of release of the odorant compounds from the sample E2 are separated in time.

11. The comparison method of claim 8, wherein the comparison is used
for studying conditions liable to influence the kinetics of release of the odorant compounds from a sample;
for determining whether sample E1 or E2 is a counterfeit; or
for quality control in the fields of perfumery, essential oils, flavors, synthetic odorant products, synthetic deodorant products, or spirits.

12. A method for generating a bank or database of temporal olfactory signatures, comprising the steps of
characterizing the kinetics of release of the odorant compounds from a plurality of samples by the method of claim 1, and obtaining a plurality of temporal olfactory signatures which characterize said samples; and
from the plurality of temporal olfactory signatures which characterize said samples, obtained in the preceding step, generating a bank or database of temporal olfactory signatures.

13. A device used in implementing the method of claim 1, comprising
(i) at least one container in which a sample is disposed;
(ii) an electronic nose having a fluidic system capable of transporting the gaseous medium comprising the odorant compounds released from said sample to the detection system of the electronic nose, which comprises a network of sensors with cross-reactivity for the odorant components present in said gaseous medium, and a computer system carrying out the processing of the responses emitted by the sensors in the form of signals;
(iii) a means for processing the signals generated by the computer system of said electronic nose so as to obtain a temporal olfactory signature; and
(iv) a means for promoting or accelerating the release of the odorant compounds from said sample.

14. The device of claim 13, wherein said means (iv) for promoting or accelerating the release of the odorant compounds from said sample takes the form of an agitator plate and/or hot plate, a heating system, an unclosed opening traversing said container and/or a forced flow system for accelerating the renewal of the gaseous medium containing the odorant compounds released from the sample.

15. The device of claim 13, further comprising a means for withdrawing from said container the gaseous medium comprising the odorant compounds released from said sample, said means being fluidically connected to the fluidic system of said electronic nose and in that a valve is installed at the fluidic connection between the withdrawal means and the fluidic system of the electronic nose and is programmed so as to pump the gaseous medium comprising the odorant compounds released from said sample at predetermined times.

16. The device of claim 13, further comprising a plurality of containers in which identical or different samples are disposed.

17. The device of claim 13, further comprising a container to which a reference composed of a pure volatile product has been added.

18. The device of claim 13, further comprising
a means for introducing air into the internal volume of the container, concomitantly with or consecutively to each withdrawal from this volume; and/or
a means for rinsing the network of sensors of the electronic nose after each contact of said nose with a withdrawal.
